(19) United States
(12) Patent Application Publication
(10) Pub. No.: US 2002/0102725 A1
(43) Pub. Date: Aug. 1, 2002

(54) Nucleic acid encoding ion transporter component protein

(76) Inventors: Eugene Zabarovsky, Upplands Vasby (SE); Alexei Protopopov, Stockholm (SE); Vladimir Kashuba, Spanga (SE)

Correspondence Address: GEMINI GENOMICS, INC., 189 WELLS AVENUE, NEWTON, MA, 02459, US

(21) Appl. No.: 09/739,754

(22) Filed: Dec. 20, 2000

(51) Int. Cl. G01N 33/53; C07H 21/04; C12P 21/06; C12N 15/00; C12N 15/09; C12N 15/63; C12N 15/70; C12N 15/74; C12N 5/00; C12N 5/02; C07K 16/00; C12P 21/08

(52) U.S. Cl. 435/325; 536/23.5; 435/320.1; 435/69.1; 435/7.1; 530/387.7

(57) ABSTRACT

The present invention provides a novel nucleic acid which encodes a protein which is a component of an ion transport system and which is expressed at high levels in human heart, brain, and kidney. The invention is further directed to the novel protein component of the ion transport system, antibodies specific for the novel protein, and assays using the novel protein as a component of an ion transport system.

This invention relates to a novel nucleic acid having high expression levels in heart, brain, and kidney, to the protein encoded by said nucleic acid, said protein having potential activity as a component of an ion transporter or ion channel, and to uses of said nucleic acid and protein in the identification and treatment of cardiovascular, neurological, or renal disorders.

### BACKGROUND OF THE INVENTION

The U.S. National Heart Lung and Blood Institute 1999 Fact Book indicates that in 1997, approximately 59.7 million Americans had cardiovascular diseases, and approximately 50 million Americans had hypertension. Approximately 12 million Americans have coronary heart disease, 4.6 million have congestive heart failure, 4 million have cerebrovascular disease, and 2 million have peripheral vascular diseases. Cardiovascular disease limits the activity of about eight million Americans. Coronary heart disease is the leading cause of death in the United States, causing 460,000 deaths in 1998. Cerebrovascular disease is the third leading cause of death in the United States, causing 158,000 deaths in 1998. The National Heart Lung and Blood Institute estimates that the economic cost of cardiovascular disease in the year 2000 will be $327 billion, in direct health expenditures and indirect costs associated with morbidity and mortality.

About a third of all known genetic defects affect the nervous system. More than 200 genes have been identified that can cause or contribute to neurological disease. For example, genes have been identified which are associated with Alzheimer's disease and Parkinson's disease, and genes have been shown to cause Duchenne muscular dystrophy, Huntington's disease, Friedreich's ataxia, Batten disease, neurofibromatosis, spinal muscular atrophy, a familial form of amyotrophic lateral sclerosis (ALS, or Lou Gehrig's disease) and several forms of epilepsy.

Many of the activities of the cardiovascular and nervous systems are mediated by active transport of ions across cell membranes and by ion-mediated intracellular signaling. Several classes of calcium channel blocking drugs are employed in treatment of cardiovascular disease, including the phenylalkylamines (e.g., verapamil), the benzothiazepines (e.g., diltiazem), and the 1,4-dihydropyridines (e.g., nifedipine). Studies are ongoing of the role of kidney ion channels and transporters in relation to renal diseases such as hypertension, such studies being directed to an epithelial sodium channel sensitive to amiloride, a sodium-chloride cotransporter sensitive to thiazide, a sodium-potassium-2chloride cotransporter sensitive to bumetanide, and a type 3 sodium-chloride exchanger,. The U.S. National Institute of Neurological Disorders has identified ion channels, synapses, and circuits as the most promising opportunities for future therapeutic breakthroughs in neurological disorders.

The flow of ions such as sodium, potassium, calcium, and chloride across external and internal cell membranes carries signals that regulate a variety of vital life processes, including muscle contraction, transmission of nerve impulses, regulation of cell volume, and the like. Ions are actively transported across cell membranes through pores known as ion channels, which are opened by ligands or changes in voltage. Moreover, when a ligand or voltage change initiates an ion channel's opening, the channel's delayed inactivation, that is, its closing, is simultaneously initiated in a regulated manner. After a recovery period, the ion channel can reopen to allow transport of more ions.

In general, ligand-gated ion channels conduct cations or anions without high selectivity, while voltage-gated ion channels are selective for a particular ion. However, pore structure, selectivity filters, and activation and inactivation gates are highly conserved across species, allowing many deductions to be made based on structure-function relationships among ion channel types. For example, the basic structure of all ion channels is a tetramic complex of a series of six α-helical transmembrane segments, connected by both intracellular and extracellular loops known as interlinkers. These α-helical segments contain the ion-conducting pore, voltage sensors, gates for opened and closed channel states, and binding sites for endogenous and exogenous ligands. The selectivity filter of an ion channel determines its ion selectivity, and substitutions in a few residues can change a pore's ion selectivity. In addition to the α-subunits, ion channels may also comprise additional, less homologous subunits, known as β-subunits, that may modify voltage sensitivity, kinetics, expression levels, or membrane localization. Usually at least two different β-subunits may bind to a single α-subunit, for example, the complete potassium channel tetramer binds up to four β₂-subunits. Some ion channels contain additional proteins, for example, calcium channels comprise two additional subunits: α₂ and δ, and in skeletal muscle and brain, also comprise a transmembrane γ-subunit.

Certain ion transporters, known as ABC transporters, form one of the largest superfamilies of proteins and examples are found in all cells from bacteria to man. Most ABC proteins are active transporters while others are ion channels. Some ABC transporters, in addition to their intrinsic transporter/channel activity, also regulate the activity of heterologous channel proteins. Many ABC proteins are of considerable clinical significance, such as the multidrug resistance P-glycoprotein which confers resistance of cancers to chemotherapy, the cystic fibrosis gene product, pfmdr which confers chloroquine resistance on the malarial parasite, and proteins in bacteria which export toxins from the cell. Combined molecular genetic, biochemical and electrophysiological techniques are necessary to address the structure, function and physiological roles of several model ABC transporters and channels. Very little information is currently available about how these membrane proteins ‘talk to each other’ to co-ordinate events within the cell membrane.

Allikmets et al. (1994) Genomics 19: 303-309 and (Zabarovsky et al., 1994) Genomics, 21: 495-500 disclose an approach combining physical and gene mapping methods to characterize large regions of human and mammalian chromosomes using NotI linking/jumping clones as framework markers. Zabarovsky et al. (1994) Genomics, 20: 312-316 and Zabarovsky et al. (2000) Nucleic Acids Res., 28: 1635-1639 discloses procedures for jumping and lining library construction and a number of chromosome 3-specific libraries and total human NotI linking libraries made using these procedures. Kashuba et al. (1999) Gene, 239: 259-271 discloses partial sequencing of more than 1,000 NotI linking clones isolated from human chromosome 3-specific libraries, in a search for a tumor suppressor gene located on chromosome 3p. Kashuba et al. further discloses that these NotI isolates constituted 152 unique chromosome 3-specific NotI clones. A search of the EMBL nucleotide database with these sequences revealed homologies (90%-100%) to more than 100 different genes or expressed sequence tags (ESTs). Many of these homologies were used to map new genes to chromosome 3.

A need continues to exist for an understanding at a molecular level of the mechanisms by which ion transporters and ion channels contribute to cardiovascular, neurological, and renal pathologies so that new diagnostic and therapeutic methodologies may be developed. One means for understanding these mechanisms is an understanding of the genetic basis for these disorders.

### SUMMARY OF THE INVENTION

The present inventors have isolated a novel human cDNA UNC93B1 (the nucleic acid sequence set forth in SEQ ID NO:2, GenBank Accession No. AJ271326) encoding a protein (the amino acid sequence set forth in SEQ ID NO:3) related to unc-93 of Caenorhabditis elegans. The combined sequence derived from several cDNA clones is 2.282 kilobase pairs and includes 11 exons. The maximal open reading frame encodes a protein of 597 amino acids, as shown in SEQ ID NO:3. Homology analysis shows that hUNC93B1 is a highly conserved cDNA related to counterparts in Arabidopsis thaliana, C. elegans, Drosophila melanogaster, chicken and mouse. Based on the structural similarity of the protein encoded by the hUNC93B1 cDNA to proteins expressed by known genes, structural analysis, and the high level of expression of the hUNC93B1 mRNA in heart, brain, and kidney, the hUNC93B1 protein may be a component of one or more ion transport systems in those tissues. Malfunction of the hUNC93B1 protein may result in cardiovascular, neurological, or renal disease.

In one embodiment, the invention provides an isolated or purified polynucleotide comprising the nucleic acid sequence set forth in SEQ ID NO:2. The invention further provides expression vectors comprising the polynucleotide of SEQ ID NO:2 in operable association with regulatory sequences which enable expression of the polynucleotide of SEQ ID NO:2 in a host cell. Host cells containing and expressing the polynucleotide of SEQ ID NO:2 are also provided.

In another embodiment, the invention provides an isolated or purified protein having an amino acid sequence as set forth in SEQ ID NO:3.

In another embodiment, the invention provides a method of identifying a drug which modulates the expression of a hUNC93B1 protein of SEQ ID NO:3, comprising the steps of contacting a host cell which expresses a polynucleotide having a sequence as set forth in SEQ ID NO:2 with a drug candidate to form an assay mixture; and detecting a decrease or increase in expression level of the hUNC93B1 protein of SEQ ID NO:3 in the assay mixture.

In yet another embodiment, the invention provides a method of identifying a drug which modulates activity of the hUNC93B1 protein of SEQ ID NO:3 as a component of an ion transport system, comprising the steps of: contacting a host cell which expresses the protein of SEQ ID NO:3 on the cell's surface with a drug candidate to form an assay mixture; and detecting a decrease or increase in ion transport activity of the ion transport system in the assay mixture.

In another embodiment, the invention provides a method of diagnosing risk or existence of a disease or disorder associated with aberrant expression or activity of the hUNC93B1 protein of SEQ ID NO:3 comprising the steps of obtaining a biological sample from a subject; combining the biological sample with an anti-hUNC93B1 antibody to form an assay mixture; and detecting the presence of the protein of SEQ ID NO:3, or proteins homologous to the protein of SEQ ID NO:3 in the assay mixture.

The invention further provides a prognostic assay or method for monitoring the effectiveness of treatment of a subject suffering from a disease or condition associated with malfunction of the hUNC93B1 polynucleotide of SEQ ID NO:2 or the hUNC93B1 protein of SEQ ID NO:3, with an agent, comprising the steps of obtaining a first biological sample from the subject prior to administration of the agent; detecting the level of expression of the protein of SEQ ID NO:3 or of a mRNA encoding the protein of SEQ ID NO:3 in the first biological sample; obtaining a second biological sample from the subject after administration of the agent; detecting the level of expression or activity of said protein or of said mRNA in the second biological sample; comparing the level of expression or activity of said protein or of said mRNA in the first biological sample with the level of expression or activity of said protein or said mRNA in the second biological sample; and altering the administration of the agent to the subject accordingly.

The prognostic assay of the invention is also embodied in a method for monitoring the effectiveness of treatment of a subject suffering from a disease or condition associated with malfunction of the hUNC93B1 polynucleotide of SEQ ID NO:2 or or the hUNC93B1 protein of SEQ ID NO:3, with an agent, comprising the steps of obtaining a first biological sample from the subject prior to administration of the agent; detecting the level of hUNC93B1-mediated ion transport activity in the first biological sample; obtaining a second biological sample from the subject after administration of the agent; detecting the level of hUNC93B1-mediated ion transport activity in the second biological sample; comparing the levels of hUNC93B1-mediated ion transport activity in the first and second biological samples; and altering the administration of the agent to the subject accordingly.

The invention is also embodied in kit comprising an anti-hUNC93B1 antibody; a detectable label, and instructions.

### BRIEF DESCRIPTION OF THE DRAWINGS

FIG. 1 shows the nuceotide and amino acid sequences of the hUNC93B1 polynucleotide (SEQ ID NO:2) and protein (SEQ ID Nos:2 and 3).

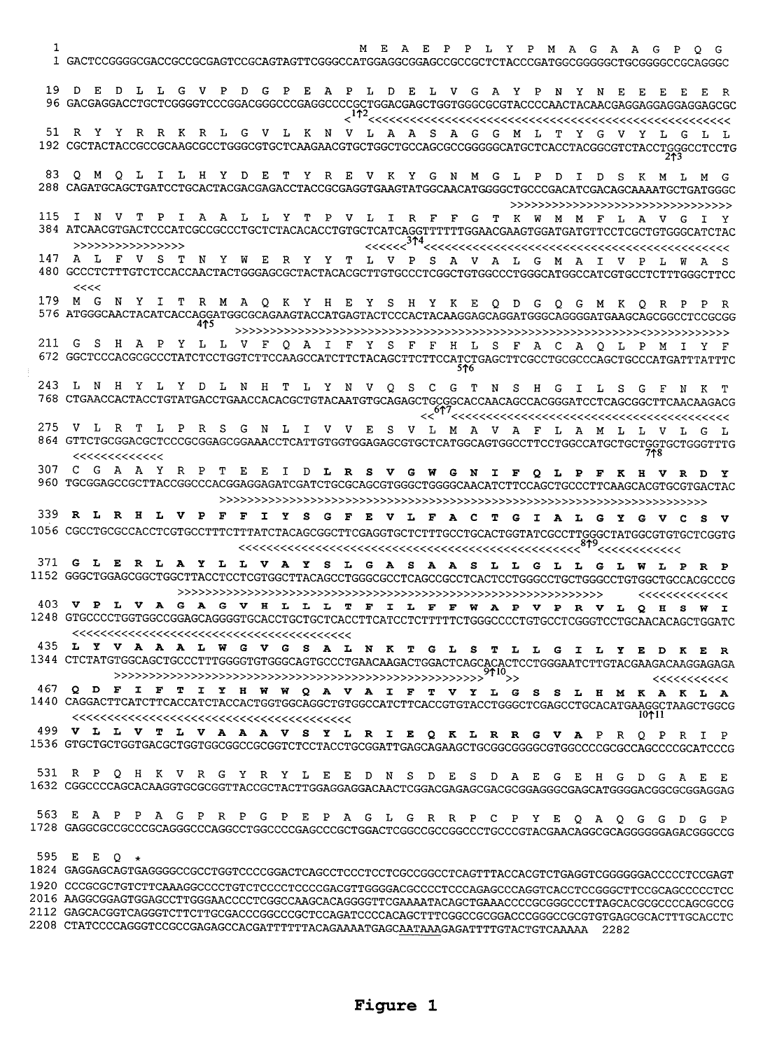

FIG. 2 shows the alignment of the predicted amino acid sequences of the family of unc-93 (C elegans) related polynucleotides. The most conserved 5′ and 3′ regions of UNC93B1 (SEQ ID NO:3) are shown (A and B, respectively).

FIG. 3 shows exon—intron organization of the hUNC93B1 polynucleotide and relationship between the hUNC93B1 polynucleotide (SEQ ID NO:2) and genomic variants similar to the 3′ portion of the hUNC93B1 polynucleotide (SEQ ID NO:2). The exact positions of exon/intron borders are shown below.

### DETAILED DESCRIPTION OF THE INVENTION

The contents of all cited references, patents and published patent applications are incorporated herein by reference.

As used herein, “ion transport activity” is defined as ligand-gated or voltage-gated flow of a cation or an anion across an intracellular or extracellular cell membrane. Cations transported in accordance with this definition include, without limitation, sodium, potassium, calcium, and zinc. Anions transported in accordance with this definition include, without limitation, chloride.

As defined herein, a “component of an ion transport system” means an ion-conducting pore, a voltage sensor, an activation gate, an inactivation gate, a selectivity filter, a binding site for an endogenous or exogenous ligand, a modifier of voltage sensitivity, a modifier of ion transport kinetics, a modifier of expression level of a protein which has a role in mediating ion transport activity, or a modifier of membrane localization of a protein or protein complex which has a role in mediating ion transport activity. The hUNC93B1 protein of SEQ ID NO:3 is a component of an ion transport system as defined herein.

As used herein, “hUNC93B1-mediated ion transport activity” means ion transport activity which is modulated or regulated, that is, increased or decreased, as the result of the interaction of the interaction of the hUNC93B1 protein of SEQ ID NO:3 with any other component of an ion transport system.

Levin and Horvitz (1992) J Cell Biol. 117: 143-155 teach that C. elegans unc-93 protein is either a component of an ion transport system involved in excitation-contraction coupling in muscle, or functions in the coordination of muscle contraction between muscle cells, by affecting the actions of gap junctions. As hUNC93B1 protein displays significant identity to C. elegans unc-93, those of ordinary skill will recognize that hUNC93B1 (SEQ ID NO:3) may have a similar function in human cells.

As indicated in Example 3 below, the highest level of expression of the hUNC93B1 mRNA (i.e., the mRNA complementary to the polynucleotide having SEQ ID NO:2) is found in heart tissue. Example 3 also indicates that expression of the hUNC93B1 mRNA is high in kidney. Thus the hUNC93B1 protein of SEQ ID NO:3 may function as a component of an ion transport system within the cardiovascular system. Malfunction in hUNC93B1 polynucleotide (SEQ ID NO:2) expression or in the hUNC93B1 protein product (SEQ ID NO:3) in the cardiovascular system may result in or contribute to symptomatology of cardiovascular disease. Exemplary cardiovascular diseases which may involve malfunction of the hUNC93B1 polynucleotide (SEQ ID NO:2) or the hUNC93B1 protein (SEQ ID NO:3) include, without limitation, atherosclerotic diseases such as coronary heart disease, that is, myocardial infarction, angina pectoris, arteriosclerosis, peripheral vascular disease, cerebrovascular disease, that is, stroke, and the like. In addition, malfunction of the hUNC93B1 polynucleotide (SEQ ID NO:2) or of the hUNC93B1 protein (SEQ ID NO:3) may contribute to conditions such as hypertension, congestive heart failure, cardiac arrythmias, renal tubular disease, renally induced polyuria, renally induced metabolic dysfunction, and the like.

Example 3 also indicates that the hUNC93B1 mRNA is expressed at high levels in brain. Thus the hUNC93B1 polynucleotide (SEQ ID NO:2) or its protein product (SEQ ID NO:3) may also function as a component of an ion transport system within the brain. Malfunction of the hUNC93B1 polynucleotide (SEQ ID NO:2) or the hUNC93B1 protein (SEQ ID NO:3) in brain may contribute to symptomatology found in such neurological disorders as Alzheimer's disease, Parkinson's disease, muscular dystrophy, Huntington's disease, ataxia, Batten disease, neurofibromatosis, spinal muscular atrophy, ALS, epilepsy, multiple sclerosis, schizophrenia, manic depressive illness, organic brain syndrome, attention deficit hyperactivity disorder, anxiety disorder, autism, migraine, and the like.

As shown in Example 4 below, the hUNC93B1 polynucleotide of the invention (SEQ ID NO:2) is located on chromosome 11q13. Locus 11q13 is associated with many diseases (Hou, et al. (1996) Hum. Hered. 46: 211-220; Katsanis, et al. (1999) Am. J. Hum. Genet. 65: 1672-1679; Lebo, et al. (1990) Hum. Genet. 86: 17-24), and some of them are connected with muscle function. An example is spinal muscular atrophy, which is associated with respiratory distress (SMARD1) (Grohmann, et al. (1999) Am. J. Hum. Genet. 65: 1459-1462). The nucleic acid and the protein of the present invention may therefore be involved in one or several of these disorders.

The hUNC93B1 polynucleotide set forth in SEQ ID NO:2 may be used in accordance with the invention for recombinant production of hUNC93B1 protein (SEQ ID NO:3) in a host cell. In this embodiment, the hUNC93B1 polynucleotide of SEQ ID NO:2 is operably linked to an expression control sequence such as an expression vector. As defined herein, “operably linked” means enzymatically or chemically ligated to form a covalent bond between the isolated polynucleotide of SEQ ID NO:2 and the expression control sequence, in such a manner that the polynucleotide of SEQ ID NO:2 is transcribed into mRNA and translated into the hUNC93B1 protein. As defined herein, “expression control sequence” includes promoters, enhancers, and other expression control elements such as those described in Goeddel, Gene Expression Technology: Methods in Enzymology 185, Academic Press, San Diego, Calif. (1990).

In accordance with the invention, any expression control sequence may be ligated to the polynucleotide of SEQ ID NO:2 to produce the hUNC93B1 protein of SEQ ID NO:3. Suitable expression vectors are commercially available, for example, from Invitrogen Corporation, San Diego, Calif., USA. Alternatively, suitable expression vectors can readily be prepared by the skilled artisan. Expression control sequences are art-recognized and are selected to produce the encoded protein in a particular host cell. In accordance with the invention, expression control sequences associated with the native hUNC93B1 polynucleotide of SEQ ID NO:2 or expression control sequences native to the transformed host cell can be employed. Those of ordinary skill will take into account that the design of the expression vector may depend on such factors as the choice of the host cell to be transformed and/or the type of protein desired to be expressed in designing a suitable expression vector for production of the hUNC93B1 protein of SEQ ID NO:3. For instance, the hUNC93B1 protein of the present invention (SEQ ID NO:3) can be produced by ligating thepolynucleotide of SEQ ID NO:2, or a portion thereof, into a vector suitable for expression in either prokaryotic cells, eukaryotic cells or both (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17). Typically, expression constructs will contain one or more selectable markers, including, but not limited to, a gene that encodes dihydrofolate reductase and genes that confer resistance to neomycin, tetracycline, ampicillin, chloramphenicol, kanamycin, streptomycin, and the like. Suitable expression systems for use in a variety of host cells are commercially available, for example, from Invitrogen Corporation, San Diego, Calif., USA.

The invention is also embodied in host cells containing the polynucleotide of SEQ ID NO:2 which are capable of expressing the protein of SEQ ID NO:3. Any host cell may be used to produce the protein of SEQ ID NO:3. For example, prokaryotic host cells of the present invention include, but are not limited to, bacterial cells such as Escherichia coli (e.g., E. coli K12 strains) Streptomyces, Pseudomonas, Serratia marcescens, Salmonella typhimurium, and the like. Eukaryotic host cells of the invention include, but are not limited to, insect cells, including Drosophila, yeast cells such as Saccharomyces cerevisiae, Schizosacchaormyces pombe, Kluyvermyces strains, Pichia strains, Candida strains, plant cells and mammalian cells, such as thymocytes, Chinese hamster ovary cells (CHO), COS cells, human kidney 293 cells, human epidermal A431 cells, human Colo205 cells, 3T3 cells, CV-1 cells, other transformed primate cell lines, normal diploid cells, cells derived from in vitro culture of primary tissue, primary explants, HeLa cells, mouse L cells, BHK cells, HL-60 cells, U937 cells, HaK cells, and the like.

The host cells of the invention may be used in cell-based screening methods for identifying drug candidates which modulate the expression of the hUNC93B1 protein of SEQ ID NO:3 or its activity as a component of an ion transport system and which thus are useful for treatment of diseases resulting from malfunction of the hUNC93B1 protein (SEQ ID NO:3). Such screening assays may be based on the ability of the drug candidate to bind to a portion of the hUNC93B1 polynucleotide of SEQ ID NO:2 or to the corresponding mRNA, thereby modulating the expression of the hUNC93B1 protein of SEQ ID NO:3. Alternatively, the screening assay of the invention may be based on the ability of the drug candidate to bind to the extracellular or intracellular portion of the hUNC93B1 protein (SEQ ID NO:3), thereby modulating, i.e., stimulating or inhibiting, the activity of the protein as a component of an ion transport system.

The screening assay of the invention comprises the steps of contacting a host cell which expresses the hUNC93B1 protein (SEQ ID NO:3) on the cell's surface with a drug candidate to form an assay mixture and determining the ability of the drug candidate to interact specifically with the hUNC93B1 polynucleotide of SEQ ID NO:2 or with the hUNC93B1 protein of SEQ ID NO:3. A specific interaction between the drug candidate and the hUNC93B1 polynucleotide or its corresponding mRNA is indicated by a decrease or increase in expression level of the hUNC93B1 protein (SEQ ID NO:3). The expression level of the hUNC93B1 protein (SEQ ID NO:3) may be determined by measuring the amount of hUNC93B1 protein (SEQ ID NO:3) in the assay mixture. Methods for making such protein measurements are known. For example, the amount of expressed hUNC93B1 protein (SEQ ID NO:3) may be measured using an antibody specific for the hUNC93B1 protein (SEQ ID NO:3) which is directly or indirectly labeled with a radioactive isotope such as ¹²⁵I, ³⁵S, ¹⁴C, or ³H, with a fluorescent molecule such as fluoroisothiocyanate, rhodamine, phycoerythrin, and the like, or with an enzyme such as horseradish peroxidase, alkaline phosphatase, or luciferase. Alternatively, the expression level of the hUNC93B1 protein (SEQ ID NO:3) may be measured by detecting the induction of a reporter gene (comprising a target-responsive regulatory element operatively linked to a nucleic acid encoding a detectable marker, e.g., luciferase) which is covalently linked to and co-expressed with the hUNC93B1 polynucleotide (SEQ ID NO:2).

Since the hUNC93B1 protein of SEQ ID NO:3 functions as a component of an ion transporter system, the protein's expression level and its activity in response to a drug candidate can be determined by measuring the amount of an ion such as calcium, sodium, potassium, or chloride transported into or out of the host cell when the cell is exposed to the drug candidate. For example, the ability of the hUNC93B1 protein of SEQ ID NO:3 to act as a component of a transporter system for a monovalent cation such as sodium or potassium may be measured using known methods based on fluorescent indicators such as those set forth in Minta, et al. (1989) J. Biol. Chem. 264, 19449-19457 and Meuwis et al. (1995) Biophys. J. 68, 2469-2473. Fluorescent dyes may be used also to measure transporter systems for divalent cations such as calcium (Fura-2, Ward, et al. (1992) J. Mol. Cell. Cardiol. 24, 937) and zinc (Zinquin (1994) Biochem. J. 303, 781), and to measure transport of monovalent anions such as chloride ion (SPQ, Mulberg, A. E., et al. (1991) J. Biol. Chem. 266, 20590). In addition, fluorescent dyes may be used to measure cell membrane potential changes, as set forth in Biochim. Biophys. Acta (1984) 771, 208).

Alternatively, the ability of a drug candidate to interact with the hUNC93B1 protein of SEQ ID NO:3 may be measured without labeling any of the interactants. For example, a microphysiometer can be used to detect the interaction of a drug candidate with the hUNC93B1 protein (SEQ ID NO:3) without labeling either the drug candidate or the protein, as set forth in McConnell, et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and receptor.

Any drug candidate may be screened for its ability to modulate the expression or ion transport activity of the hUNC93B1 protein of SEQ ID NO:3. Drug candidates may be obtained using any of the numerous approaches in combinatorial library methods known in the art, including: biological libraries; spatially addressable parallel solid phase or solution phase libraries; synthetic library methods requiring deconvolution; the one-bead one-compound library method; and synthetic library methods using affinity chromatography selection. See, e.g, Lam, K. S. (1997) Anticancer Drug Des. 12:145; DeWitt, et al. (1993) Proc. Natl. Acad. Sci. U.S.A. 90:6909; Erb, et al. (1994) Proc. Natl. Acad. Sci. U.S.A. 91:11422; Zuckermann, et al. (1994). J. Med. Chem. 37:2678; Cho, et al. (1993) Science 261:1303; Carell, et al. (1994) Angew. Chem. Int. Ed. Engl. 33:2059; Carell, et al. (1994) Angew. Chem. Int. Ed. Engl. 33:2061; and in Gallop, et al. (1994) J. Med. Chem. 37:1233.

Libraries of drug candidates may be presented in solution (e.g., Houghten (1992) Biotechniques 13:412-421), or on beads (Lam(1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. U.S.A. 89:1865-1869) or on phage (Scott and Smith (1990) Science 249:386-390); (Devlin (1990) Science 249:404-406); (Cwirla, et al. (1990) Proc. Natl. Acad. Sci. U.S.A. 97:6378-6382); (Felici (1991) J. Mol. Biol. 222:301-310); (Ladner. supra).

In yet another aspect of the invention, the proteins of the invention can be used as “bait proteins” in a two-hybrid assay or three-hybrid assay (see, e.g., U.S. Pat. No. 5,283,317; Zervos, et al. (1993) Cell 72:223-232; Madura, et al. (1993) J. Biol. Chem., 268:12046-12054; Bartel, et al. (1993) Biotechniques 14:920-924; Iwabuchi, et al. (1993) Oncogene 8:1693-1696; and Brent in W094/10300), to identify other proteins (captured proteins) which bind to or interact with the hUNC93B1 protein of the invention (SEQ ID NO:3) and modulate its activity. Such captured proteins are also likely to be involved in the propagation of signals by the hUNC93B1 protein of SEQ ISD NO:3 as, for example, downstream elements of a protein-mediated signaling pathway. Alternatively, such captured proteins are likely to be cell-surface molecules associated with non-protein-expressing cells, wherein such captured proteins are involved in signal transduction.

The two-hybrid system is based on the modular nature of most transcription factors, which consist of separable DNA-binding and activation domains. Briefly, the assay utilizes two different DNA constructs. In one construct, the hUNC93B1 polynucleotide of SEQ ID NO:2 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected, and cell colonies containing the functional transcription factor can be isolated and used to obtain the cloned gene which encodes the protein which interacts with the protein of the invention.

The hUNC93B1 polynucleotide of SEQ ID NO:2 and the hUNC93B1 protein of SEQ ID NO:3 can be isolated or purified from recombinant cell culture by a variety of processes. As used herein, the term “isolated” means that at least 75% of the cellular components other than the hUNC93B1 polynucleotide of SEQ ID NO:2 or the hUNC 93B1 protein of SEQ ID NO:3 have been removed from the solution containing the hUNC93B1 polynucleotide of SEQ ID NO:2 or the hUNC 93B1 protein of SEQ ID NO:3. The term “purified” means that at least 85% of the cellular components other than the hUNC93B1 polynucleotide of SEQ ID NO:2 or the hUNC93B1 protein of SEQ ID NO:3 have been removed from the solution containing the hUNC93B1 polynucleotide of SEQ ID NO:2 or the hUNC 93B1 protein of SEQ ID NO:3. Methods for isolating or purifying the hUNC93B1 polynucleotide of SEQ ID NO:2 or the hUNC93B1 protein of SEQ ID NO:3 include, but are not limited to, membrane filtration, anion or cation exchange chromatography, ethanol precipitation, affinity chromatography, high performance liquid chromatography (HPLC), and the like. The particular method used will depend upon the properties of the particular form of the hUNC93B1 polynucleotide of SEQ ID NO:2 or the hUNC93B1 protein of SEQ ID NO:3 to be isolated and the selection of the host cell; appropriate methods will be readily apparent to those skilled in the art. For example, it may be desirable to isolate a solubilized form of the hUNC93B1 protein of SEQ ID NO:3 for a particular study, and to accomplish this a solubilizing agent is used such as the non-ionic detergents n-octylglucoside, n-dodecylglucoside, n-dodecylmaltoside, octanoyl-N-methylglucamide, decanoyl-N-methylglucamide, Triton®X-100, Triton®X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]2-hydroxy-1-propane sulfonate (CHAPSO), N-dodecyl-N, dimethyl-3-ammonio- 1-propane sulfonate, and the like.

The isolated or purified hUNC93B1 protein of SEQ ID NO:3 may be used a cell-free assay in which the protein is contacted with a drug candidate and the ability of the drug candidate to bind to the hUNC93B1 protein of SEQ ID NO:3 or to modulate the activity of the hUNC93B1 protein of SEQ ID NO:3 as a component of an ion transport system is determined. Binding of the drug candidate to the hUNC93B1 protein of SEQ ID NO:3 or modulation of the hUNC93B1 protein's (SEQ ID NO:3) activity as a component of an ion transport system can be determined either directly or indirectly as described above. Determining the ability of the protein to bind to a target molecule can also be accomplished using a technology such as real-time Bimolecular Interaction Analysis (BIA). Sjolander, S. and Urbaniczky, C. (1991) Anal. Chem. 63:2338-2345 and Szabo, et al. (1995) Curr. Opin. Struct. Biol. 5:699-705. As used herein, “BIA” is a technology for studying biospecific interactions in real time, without labeling any of the interactants (e.g., BIAcore™.). Changes in the optical phenomenon surface plasmon resonance (SPR) can be used as an indication of real-time reactions between biological molecules.

The cell-free assay of the present invention is amenable to use of both soluble and/or membrane-bound forms of the isolated hUNC93B1 protein of SEQ ID NO:3. In the assay methods of the invention, it may be desirable to immobilize either the hUNC93B1 protein of SEQ ID NO:3 or the drug candidate to facilitate separation of complexed from uncomplexed forms of the protein, as well as to accommodate automation of the assay. Binding of a drug candidate to the hUNC93B1 protein of SEQ ID NO:3, or interaction of the hUNC93B1 protein of SEQ ID NO:3 with a target molecule in the presence and absence of a drug candidate, can be accomplished in any vessel suitable for containing the reactants. Examples of such vessels include microtitre plates, test tubes, micro-centrifuge tubes, and the like. In one embodiment, a fusion protein can be provided which adds a domain that allows the hUNC93B1 protein of SEQ ID NO:3 to be bound to a matrix. For example, glutathione-S-transferase fusion proteins can be adsorbed onto glutathione sepharose beads (Sigma Chemical, St. Louis, Mo., USA) or glutathione derivatized microtitre plates, which are then combined with the drug candidate or the drug candidate and the non-adsorbed hUNC93B1 protein of the invention (SEQ ID NO:3), and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, either a hUNC93B1 protein of the invention (SEQ ID NO:3) or a drug candidate can be immobilized utilizing conjugation of biotin and streptavidin. Biotinylated protein of the invention or drug candidates can be prepared from biotin-NHS(N-hydroxysuccinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the hUNC93B1 protein of SEQ ID NO:3, but which do not interfere with binding of the protein to a drug candidate, can be derivatized to the wells of the plate, and unbound hUNC93B1 protein (SEQ ID NO:3) can be trapped in the wells by virtue of its interaction with the antibody. Methods for detecting such complexes, in addition to those described above for the GST-immobilized complexes, include immunodetection of complexes using antibodies reactive with the hUNC93B1 protein of SEQ ID NO:3, as well as ion channel-based assays which rely on detecting hUNC93B1-mediated ion transport activity.

The isolated or purified hUNC93B1 protein of SEQ ID NO:3 may also be used to generate polyclonal and monoclonal antibodies specific thereto. The antibodies of the invention include non-human and human antibodies, humanized antibodies, chimeric antibodies and antigen-binding fragments thereof (Current Protocols in Immunology, John Wiley & Sons, N.Y. (1994); EP Application 173,494; International Patent Application W086/01533; and U.S. Pat. No. 5,225,539) which bind to the hUNC93B1 protein of SEQ ID NO:3. To generate such antibodies, a mammal, such as a mouse, rat, hamster or rabbit, can be immunized with an immunogenic form of the hUNC93B1 protein (e.g., the full length hUNC93B1 protein of SEQ ID NO:3 or a polypeptide comprising an antigenic fragment of the hUNC93B1 protein which is capable of eliciting an antibody response). Techniques for conferring immunogenicity on a protein or polypeptide are well known in the art, and include such methods as conjugation of the protein or polypeptide to any of a variety of carriers or administration of the protein or polypeptide with an adjuvant. The progress of immunization can be monitored by detection of antibody titers in plasma or serum. Standard ELISA or other immunoassays can be used with the immunogen as antigen to assess the levels of antibody.

Following immunization, anti-peptide antisera can be obtained, and if desired, polyclonal antibodies can be isolated from the serum. Monoclonal antibodies can also be produced by standard techniques which are well known in the art (Kohler and Milstein, Nature 256:495-497 (1975); Kozbar, et al., Immunology Today 4:72 (1983); and Cole, et al., Monoclonal Antibodies and Cancer Therapy, Alan R. Liss, Inc., pp. 77-96 (1985)). The term “antibody” as used herein is intended to include fragments thereof, such as Fab and F(ab′)₂.

The anti-hUNC93B1 antibodies of the invention can be used in binding assays of the hUNC93B1 protein, particularly in vitro assays of cells or cell extracts, using methods known in the art. Additionally, such antibodies, in conjunction with a label, such as a radioactive label, can be used to assay for the presence or amount of the expressed hUNC93B1 protein in a cell in the screening assays described above or from a biological sample such as heart, brain, or kidney tissue in a diagnostic assay. The anti-hUNC93B1 antibodies of the invention can also be used in an immunoabsorption process, such as an immunoadsorbent column, to isolate the hUNC93B1 protein of SEQ ID NO:3 or homologous proteins from biological samples. In labeled form, the anti-hUNC93B1 antibodies of the invention are also useful in antibody-based diagnostic assays of cardiac or renal function, for example, radioimmunoassays, enzyme-linked immunosorbant assays, fluorescence-based immunoassays, and the like.

The invention is also embodied in diagnostic methods used to identify subjects having or at risk of developing a disease or disorder associated with aberrant expression of the hUNC93B1 protein of SEQ ID NO:3 with or the activity of the hUNC93B1 protein of SEQ ID NO:3 as a component of an ion transport system. For example, the assays described herein can be employed to identify a subject having or at risk of developing a cardiovascular, neurological, or renal disorder associated with hUNC93B1 protein (SEQ ID NO:3) expression or activity. Such disorders may include, without limitation, atherosclerotic diseases such as coronary heart disease, that is, myocardial infarction, angina pectoris, arteriosclerosis, peripheral vascular disease, cerebrovascular disease, that is, stroke, hypertension, congestive heart failure, cardiac arrythmias, renal tubular disease, renally induced polyuria, renally induced metabolic dysfunction, Alzheimer's disease, Parkinson's disease, muscular dystrophy, Huntington's disease, ataxia, Batten disease, neurofibromatosis, spinal muscular atrophy, ALS, epilepsy, multiple sclerosis, schizophrenia, manic depressive illness, organic brain syndrome, attention deficit hyperactivity disorder, anxiety disorder, autism, migraine, and the like.

In the diagnostic assay of the invention, a biological sample is obtained from a subject. As used herein, “biological sample” means a tissue, blood, serum, plasma, or other biological fluid sample. Using the anti-hUNCB1 antibody of the invention, hUNC93B1 protein of SEQ ID NO:3, or proteins homologous to the hUNC93B1 protein of SEQ ID NO:3, is detected, wherein the presence of hUNC93B1 protein of SEQ ID NO:3 or proteins homologous to the hUNC93B1 protein of SEQ ID NO:3 is diagnostic for risk or existence of a disease or disorder associated with aberrant expression or activity of the hUNC93B1 protein of SEQ ID NO:3.

The invention also encompasses a prognostic assay, that is, a method for monitoring the effectiveness of treatment of a subject suffering from a disease or condition associated with malfunction of the hUNC93B1 polynucleotide of SEQ ID NO:2 or the hUNC93B1 protein of SEQ ID NO:3, with an agent (e.g., an agonist, antagonist, peptidomimetic, protein, polypeptide, nucleic acid, small molecule, or other drug candidate identified by the screening assays described herein). The prognostic assay of the invention comprises the steps of a) obtaining a first biological sample from a subject prior to administration of the agent; b) detecting the level of expression of the hUNC93B1 protein of SEQ ID NO:3 or of the mRNA encoding the hUNC93B1 protein of SEQ ID NO:3, in the first biological sample; c) obtaining a second biological sample from the subject after administration of the agent; d) detecting the level of expression or activity of the protein of SEQ ID NO:3 or of the mRNA encoding the hUNC93B1 protein of SEQ ID NO:3 in the second biological sample; e) comparing the level of expression or activity of the hUNC93B1 protein of SEQ ID NO:3 or of the mRNA encoding the hUNC93B1 protein of SEQ ID NO:3 in the first biological sample with the level of expression of activity of the hUNC93B1 protein of SEQ ID NO:3 or of the mRNA encoding the hUNC93B1 protein of SEQ ID NO:3 the second biological sample; and f) altering the administration of the agent to the subject accordingly.

Alternatively, the prognostic assay of the invention comprises the steps of: a) obtaining a first biological sample from a subject prior to administration of the agent; b) detecting the level of hUNC93B1-mediated ion transport activity in the first biological sample; c) obtaining a second biological sample from the subject after administration of the agent; d) detecting the level of hUNC93B1-mediated ion transport activity in the second biological sample; e) comparing the levels of hUNC93B1- mediated ion transport activities in the first and second biological samples; and f) altering the administration of the agent to the subject accordingly. In the prognostic assays of the invention, “altering” the administration of the agent encompasses either increasing or decreasing the amount of agent administered, a step which is ultimately decided by the attending physician, taking into account the nature and severity of the condition being treated, and the nature of prior treatments which the subject has undergone.

The anti-hUNC93B1 antibodies may be used in kit form for detecting the presence of the hUNC93B1 protein of SEQ ID NO:3 or cross-reactive homologous proteins in a biological sample. For example, the kit can comprise a labeled or unlabeled anti-hUNC93B1 antibody; optionally, a labeled second antibody; instructions; optionally, buffers; optionally, test tubes, microtitre plates, or other items to facilitate use of the diagnostic method. The components of the kit can be packaged in a suitable container.

The examples set forth below describe the isolation and characterization of the hUNC93B1 polynucleotide of SEQ ID NO:2 and the hUNC93B1 protein of SEQ ID NO:3 and are not intended to limit the scope of the invention as described herein.

### EXAMPLE 1

### Isolation of hUNC93B1 cDNA

NotI linking clones were isolated from NotI linking libraries described in Zabarovsky et al. (1994) Genomics, 20: 312-316. The NotI linking clone NL1-304 (SEQ ID NO:1, D3S4632, GenBank Accession Nos. AJ272058, AJ272059) maps to chromosome 3p12-p13 and showed 97% identity over 40 bp to a human EST clone (GenBank Accession No. AA632247 (SEQ ID NO:4)). Using a combination of different methods, a 2282 bp cDNA sequence was identified (SEQ ID NO:2).

Specifically, a cDNA library from heart (Stratagene, La Jolla, Calif., USA) in λ ZAP II was used for the screening and isolation of cDNA clones. Growth of λ phages and plasmids, DNA isolation and other general microbiology and molecular biology methods were performed according to standard procedures (Sambrook et al. (1989) Molecular Cloning: A Laboratory Manual. Cold Spring Harbour Laboratory Press, Cold Spring Harbor, N.Y.). Marathon-Ready™ cDNA from skeletal muscle (Clontech, Palo Alto, Calif., USA) was used for 5′- and 3′-RACE PCR. Sequencing was performed using an ABI310 sequencer (Perkin Elmer, Foster City, Calif.) according to the manufacturer's instructions. Sequence assembling was done using DNASIS (HITACHI-Pharmacia).

The cDNA of SEQ ID NO:2 encodes a maximal open reading frame of 597 amino acids (SEQ ID NOs:2 and 3). The predicted molecular weight of the protein of SEQ ID NO:3 is 66.6 kDa.

### EXAMPLE 2

### Functional Analysis of hUNC93B1 Polynucleotide and Protein

DNA homology searches were performed using BLASTX and BLASTN (Altschul et al. (1990) J. Mol. Biol., 215: 403-410; Gish and States (1993) Nat. Genet., 3: 266-272) programs at the NCBI server: http://www.ncbi.nlm.nih.gov:80/BLAST. The BEAUTY Post-Processor was used with the BLASTP protein databases searches provided by the Human Genome Sequencing Center (Houston, Tex.): http://dot.imgen.bcm.tmc.edu: 9331. Scanning the PROSITE and the PfamA protein families and domains was performed at the server of the Swiss Institute for Experimental Cancer Research: http://www.isrec.isb-sib.ch/software/PFSCAN_form.html. Multiple sequence alignment was done by ClustalW program: http://www.clustalw.genome.ad.jp. The prediction of possible transmembrane regions and their orientation (TMpred prediction) was provided by the ISREC-server: www.ch.embnet.org. The algorithm of TMpred program is based on the statistical analysis of TMbase, a database of naturally occuring transmembrane proteins. The prediction was made using a combination of several weight-matrices for scoring (Hofmann and Stoffel (1993) Biol. Chem. 347: 166).

BLASTX comparison using the 597 amino acid sequences of SEQ ID NO:3 revealed significant similarities to C. elegans unc-93 protein (21% identity over 487 amino acids, expected E=10⁻¹⁹; GenBank Accession Nos. Z81449, X64415). Table 1 shows homologies among various proteins related to C. elegans unc-93 protein, including the hUN93B1 protein of SEQ ID NO:3. In Table 1, NSS indicated that no significant similarity was found

TABLE 1

| | Human | | | | | | | | |
|---|---|---|---|---|---|---|---|---|---|
| | homolog | Mouse | Chicken | Human | | | | | |
| | B1, | homolog | homolog | homolog | | D. | | D. | |
| | hUNC93B | B, | B, | A, | C. elegans | melanogaster | A. thaliana | radiodurans | H. pylori |
| GENES | 1 | mUnc93b | cUnc93b | hUNC93A | unc93 | AF145657 | AC016661 | AAF12127 | O05731 |
| Human homolog | | 86% 118aa | 62% 275aa | 28% 140aa | 21% 487aa | 22% 478aa | 31% 63aa | 27% 224aa | 31% 64aa |
| B1, hUNC93B1 | | 84% 355aa | 83% 207bp | 28% 203aa | | | | | |
| | | | 77% 322bp | | | | | | |
| Mouse homolog | 86% 118aa | | 60% 116aa | 28% 106aa | 26% 97aa | 23% 96aa | NSS | 40% 52aa | 40% 49aa |
| B, | 84% 355bp | | | | | | | | |
| mUnc93b | | | | | | | | | |
| Chicken | 62% 275aa | 60% 116aa | | 26% 198aa | 27% 157aa | 26% 186aa | NSS | NSS | NSS |
| homolog B, | 83% 207bp | | | | | | | | |
| cUnc93b | 77% 322bp | | | | | | | | |
| Human homolog | 28% 140aa | 28% 106aa | 26% 198aa | | 37% 174aa | 43% 238aa | 32% 158aa | NSS | NSS |
| A, hUNC93A | 28% 203aa | | | | 36% 176aa | 37% 211aa | 42% 54aa | | |
| C. elegans | 21% 487aa | 26% 97aa | 27% 157aa | 37% 174aa | | 35% 444aa | 34% 135aa | NSS | 30% 36aa |
| unc93 | | | | 36% 176aa | | | 37% 61aa | | |
| D. melanogaster | 22% 478aa | 23% 96aa | 26% 186aa | 43% 238aa | 35% 444aa | | 25% 314aa | NSS | NSS |
| AF145657 | | | | 37% 211aa | | | | | |
| A. thaliana | 31% 63aa | NSS | NSS | 32% 158aa | 34% 135aa | 25% 314aa | | NSS | NSS |
| AC016661 | | | | 42% 54aa | 37% 61aa | | | | |
| D. radiodurans | 27% 224aa | 40% 52aa | NSS | NSS | NSS | NSS | NSS | | 41% 285aa |
| AAF12127 | | | | | | | | | |
| H. pylori | 31% 64aa | 40% 49aa | NSS | NSS | 30% 36aa | NSS | NSS | 41% 285aa | |
| O05731 | | | | | | | | | |

### EXAMPLE 3

### Expression Analysis of the hUNC93B1 Transcript

Northern blot analysis was performed using the cDNA clone AA632247 (SEQ ID NO:4) as a probe for hUNC93B1 expression in different human tissues. Hybridization with MTN Northern filter (Clontech, Palo Alto, Calif., USA) was done according to the manufacturer's protocols. One transcript of approximately 2.4 kb was expressed in all tissues tested, although the level of the expression varied very significantly. Expression was highest in the heart and lowest in placenta. Expression of hUNC93B1 was also extremely high in brain and kidney.

After analysis of seven 5′ EST clones existing in public databases, in two of them (EST clones AA632247 (SEQ ID NO:4) and AW844512) the structure of the mRNA is changed as a result of alternative or incomplete splicing. The intron located between exons 4 and 5 is present in these clones, resulting in the creation of a termination codon (TGA) at amino acid position 186.

### EXAMPLE 4

### Chromosomal Localization of hUNC93B1

The standard procedure of FISH analysis with metaphase chromosomes was performed as described in Protopopov et al. (1996) Chromosome Res. 4: 443-447. About 60 metaphases were analyzed for each probe.

NLI-304 (SEQ ID NO:1) displays 97% identity over 40 base pairs (bp) to a human EST clone (GenBank Accession No. AA632247, SEQ ID NO:4). Using FISH, AA632247 (SEQ ID NO:4) was mapped to chromosomal site 11q13. Using FISH, the NotI linking clone NR5-KE20 (SEQ ID NO:5) was localized to four different chromosomal bands: 3p12-p13, 4p16, 7p22 and 11q13. Clone NL1-304 (SEQ ID NO:1) and a genomic probe containing hUNC93B1 exons 1-8 (introns 1-7) showed the same distribution in contrast to the EST clone AA632247 (SEQ ID NO:4) that mapped to 11q13 only.

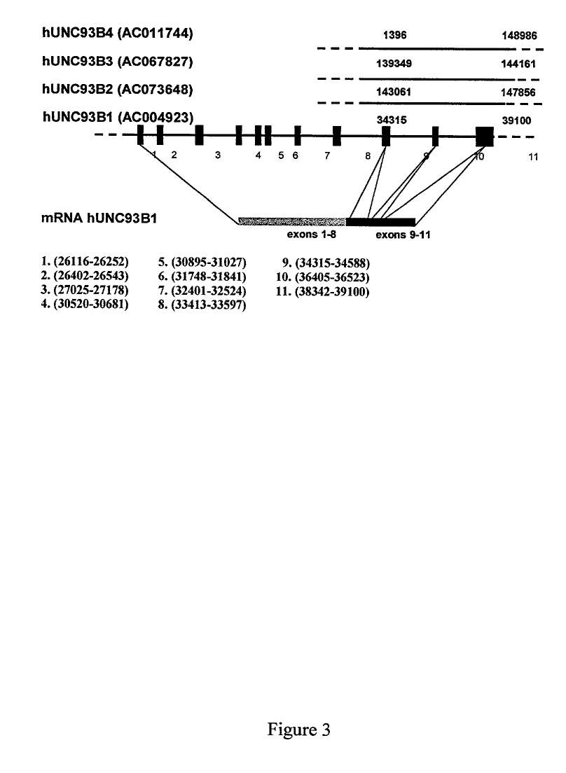

Because NR5-KE20 (SEQ ID NO:5) and NL1-304 (SEQ ID NO:1) mapped to several chromosomal locations, and because the human genome contains highly similar but not identical sequences, it is likely that hUNC93B1 (SEQ ID NO:2) is a member of a family of related genes. Based on the premise that hUNC93B1 (SEQ ID NO:2) is located in 11q13 within the PAC clone RP5-901A4 and BAC clone RP11-138N3, 11 exons can be identified, the locations of which are shown in FIG. 3.

A search with the hUNC93B1 nucleotide sequence in the EMBL and EST databases resulted in the identification of three groups of highly (95%-100%) homologous human sequences:

1. NotI linking clones:

a) NL1-304 (SEQ ID NO:1) isolated from a chromosome 3-specific library (Zabarovsky et al., 1994b) showed identity 95% over 376 bp

b) NR5-KE20 (SEQ ID NO:5; GenBank Accession Nos. AJ272060, AJ272061, 97.5% identity over 466 bp)

2. BAC and PAC clones:

a) RP11-138N3 (GenBank Accession No. AC034259) mapped to chromosome 11 (identity 99%-100% exons from 1 to 7 and exons 10, 11)

b) RP11-413E6 (GenBank Accession No. AC012661), mapped to chromosome 18 (identity 96% over 274 bp, 99% over 119 bp and 95% over 736 bp)

c) CTD-2026G6 (GenBank Accession No. AC067827), mapped to chromosome 3 (identity 96% over 274 bp, 99% over 119 bp and 95% 761 bp)

d) RP 11-747H12 (GenBank Accession No. AC073648), mapped to chromosome 7 (identity 93% over 274 bp, 100% over 119 bp and 95% over 758 bp bp)

e) RP5-901A4 (GenBank Accession No. AC004923), without localization (identity 99-100% to the whole hUNC93B1 sequence)

f) RP11-324110 (GenBank Accession No. AC011744), mapped to chromosome 4 (identity 93% over 274 bp, 97% over 119 bp and 96% over 181 bp)

3. Numerous (more than 100) unmapped ESTs (identity 94-95%).

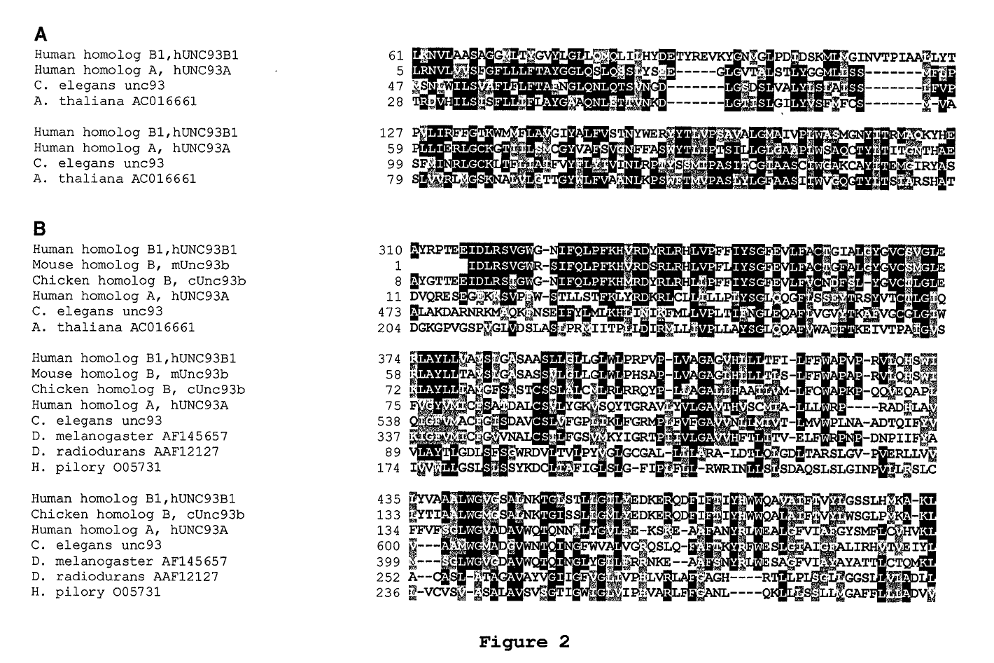

As shown in FIG. 3, a number of these human sequences have identity to the 3′ part of the hUNC93B1 (exons 9-11). Genomic (including introns) sequences of the PAC and BAC clones are very similar in this region. The most probable explanation is that in other cases sequences for 5′ ends of the respective genes are not yet known. However, it is also possible that the homologous sequences do not have this 5′ end at all and that the 3′ part of the hUNC93B1 (SEQ ID NO:2) can exist as a separate gene. The 5′ end of the hUNC93B1 (exons 1-8) is similar to that of unc93 (FIG. 2A).

Those skilled in the art will recognize, or will be able to ascertain using no more than routine experimentation, numerous equivalents to the specific substances and procedures described herein. Such equivalents are considered to be within the scope of this invention, and are encompassed by the following claims.

1. An isolated or purified polynucleotide having a nucleic acid sequence as set forth in SEQ ID NO:2.

2. The polynucleotide of claim 1, further comprising an expression control sequence in operable association with the polynucleotide.

3. A host cell comprising the polynucleotide of claim 2.

4. The cell of claim 3, wherein the cell is a prokaryotic cell.

5. The cell of claim 3, wherein the cell is a eukaryotic cell.

6. The cell of claim 5, wherein the cell is a mammalian cell.

7. An isolated or purified protein having an amino acid sequence as set forth in SEQ ID NO:3.

8. A method of identifying a drug which modulates the expression of a hUNC93B1 protein of SEQ ID NO:3, comprising the steps of:
a) contacting a host cell which expresses a polynucleotide having a sequence as set forth in SEQ ID NO:2 with a drug candidate to form an assay mixture; and
b) detecting a decrease or increase in expression level of the hUNC93B1 protein of SEQ ID NO:3 in the assay mixture.

9. A method of identifying a drug which modulates the activity of a hUNC93B1 protein having an amino acid sequence as set forth in SEQ ID NO:3 in an ion transport system, comprising the steps of:
a) contacting a host cell which expresses the hUNC93B1 protein (SEQ ID NO:3) on the cell's surface with a drug candidate to form an assay mixture; and
b) detecting a decrease or increase in the ion transport activity associated with the hUNC93B1 protein in the assay mixture.

10. A method of diagnosing risk or existence of a disease or disorder associated with aberrant expression of a hUNC93B1 protein having an amino acid sequence as set forth in SEQ ID NO:3, comprising the steps of:
a) obtaining a biological sample from a subject;
b) combining the biological sample with an anti-hUNC93B1antibody; and
c) detecting the presence of hUNC93B1 protein of SEQ ID NO:3, or proteins homologous to the hUNC93B1 protein of SEQ ID NO:3.

11. A method for monitoring the effectiveness of a treatment of a subject suffering from a disease or condition associated with a malfunction of a hUNC93B1 polynucleotide having a nucleotide sequence as set forth in SEQ ID NO:2 or a hUNC93B1 protein having an amino acid sequence as set forth in SEQ ID NO:3, with an agent, said method comprising the steps of:
a) obtaining a first biological sample from a subject prior to administration of the agent;
b) detecting the level of expression of the hUNC93B1 protein of SEQ ID NO:3 or of an mRNA encoding the protein of SEQ ID NO:3 in the first biological sample;
c) obtaining a second biological sample from the subject after administration of the agent;
d) detecting the level of expression or activity of said protein or said mRNA in the second biological sample;
e) comparing the level of expression or activity of said protein or said mRNA in the first biological sample with the level of expression or activity of said protein or said mRNA in the second biological sample; and
f) altering the administration of the agent to the subject accordingly.

12. A method for monitoring the effectiveness of a treatment of a subject suffering from a disease or condition associated with a malfunction of the hUNC93B1 polynucleotide having a nucleic acid sequence as set forth in SEQ ID NO:2 or of a hUNC93B1 protein having an amino acid sequence as set forth in SEQ ID NO:3, with an agent, said method comprising the steps of:
a) obtaining a first biological sample from a subject prior to administration of the agent;
b) detecting the level of hUNC93B1-mediated ion transport activity in the first biological sample;
c) obtaining a second biological sample from the subject after administration of the agent;
d) detecting the level of hUNC93B1-mediated ion transport activity in the second biological sample;
e) comparing the levels of hUNC93B1-mediated ion transport activity in the first and second biological samples; and
f) altering the administration of the agent to the subject accordingly.

13. An antibody specific for a protein having an amino acid sequence as set forth in SEQ ID NO:3.

14. The antibody of claim 13, wherein the antibody is a polyclonal antibody.

15. The antibody of claim 13, wherein the antibody is a monoclonal antibody.

16. A kit comprising the antibody of any one of claims 13 through 15; a detectable label, and instructions.


SEQUENCE LISTING (5 sequences)

SEQ ID NO: 1 - DNA, 1275 bp, homo sapiens
ctctgaggcttaggcacgagaatggcttgacctagagatgaggttgcagtgagccaagatcgcgccactgcacttcagcctggaagggaccagaagcgagaccctgtctcccaaaaaaaagaaaaaaagaaaaaagaaaagcagtgagtgggcagggcatggtggctcatgcctgtaatcccaacactttgggaggctgacgcaggaggattgcttgaggccaggagttcaagaccagcctgggcaacataagagaccctgcctctacaaaaaatttaaaaattagctgggagtggtggcgcgtgcctgtagttccagctacttgggagactgaggtgggaggatggcttgagcctcaaagattgaggctgaagtgagcatgccactgcgctccagcagtgggtggggggagggagggagggggagcggtggggaaacggagcgaccgtgtctcgaaaaaagaaaatagccggaagtatgcatacagatatgtgtgtatgtactgagctatgttgtaaaaatcattcctgactgcgggttatagtcaaaaccccatgaaaagcatcactacagcccacgggtgtgtcagggactcagtgttgtgagccctgggaaggcagggcctgtggccagcactttatcaacactggcacatgcaccctatgaggcaaagggatttgcattgtctccttacagcgtgggacactgaggtcgccaggggcatggtgactgtaagggacagtgctggatgtgagcctcgcctgcaggaggcggtccaggaagagtgggtggaggggctggagaagttgagggccgcgtggcccgggaggctcccggaggagggaagggcctatctcagcgaggggcataggcgggaaaggtgtggggcgaggcggccgcgggtccctggcatccctctccttacgcccaggctaagctggcgttgctgctggtgacgctggtggcggccacggtctcctacctgcggatggagcagaagctgcggcggggcgtggccccgcggcagccccgcatcccgcggccccagcacaaggtgcgcggttaccgctacttggaggaggacaactcggacgagagcgacgcggagggcgatcttggggacggcgaggaggcggaggcggaggctccgcccgcagggcccaggcctggccccgagcccgctggactcggccgccggccctgcccgtacgaacaggcgcaggggggcgatgggccggaggagcagtgaggggccgcctggtccccggactcagc

SEQ ID NO: 2 - DNA, 2282 bp, homo sapiens
  CDS (42)..(1832)
gactccggggcgaccgccgcgagtccgcagtagttcgggccatggaggcggagccgccgctctacccgatggcgggggctgcggggccgcagggcgacgaggacctgctcggggtcccggacgggcccgaggccccgctggacgagctggtgggcgcgtaccccaactacaacgaggaggaggaggagcgccgctactaccgccgcaagcgcctgggcgtgctcaagaacgtgctggctgccagcgccgggggcatgctcacctacggcgtctacctgggcctcctgcagatgcagctgatcctgcactacgacgagacctaccgcgaggtgaagtatggcaacatggggctgcccgacatcgacagcaaaatgctgatgggcatcaacgtgactcccatcgccgccctgctctacacacctgtgctcatcaggttttttggaacgaagtggatgatgttcctcgctgtgggcatctacgccctctttgtctccaccaactactgggagcgctactacacgcttgtgccctcggctgtggccctgggcatggccatcgtgcctctttgggcttccatgggcaactacatcaccaggatggcgcagaagtaccatgagtactcccactacaaggagcaggatgggcaggggatgaagcagcggcctccgcggggctcccacgcgccctatctcctggtcttccaagccatcttctacagcttcttccatctgagcttcgcctgcgcccagctgcccatgatttatttcctgaaccactacctgtatgacctgaaccacacgctgtacaatgtgcagagctgcggcaccaacagccacgggatcctcagcggcttcaacaagacggttctgcggacgctcccgcggagcggaaacctcattgtggtggagagcgtgctcatggcagtggccttcctggccatgctgctggtgctgggtttgtgcggagccgcttaccggcccacggaggagatcgatctgcgcagcgtgggctggggcaacatcttccagctgcccttcaagcacgtgcgtgactaccgcctgcgccacctcgtgcctttctttatctacagcggcttcgaggtgctctttgcctgcactggtatcgccttgggctatggcgtgtgctcggtggggctggagcggctggcttacctcctcgtggcttacagcctgggcgcctcagccgcctcactcctgggcctgctgggcctgtggctgccacgcccggtgcccctggtggccggagcaggggtgcacctgctgctcaccttcatcctctttttctgggcccctgtgcctcgggtcctgcaacacagctggatcctctatgtggcagctgccctttggggtgtgggcagtgccctgaacaagactggactcagcacactcctgggaatcttgtacgaagacaaggagagacaggacttcatcttcaccatctaccactggtggcaggctgtggccatcttcaccgtgtacctgggctcgagcctgcacatgaaggctaagctggcggtgctgctggtgacgctggtggcggccgcggtctcctacctgcggattgagcagaagctgcggcggggcgtggccccgcgccagccccgcatcccgcggccccagcacaaggtgcgcggttaccgctacttggaggaggacaactcggacgagagcgacgcggagggcgagcatggggacggcgcggaggaggaggcgccgcccgcagggcccaggcctggccccgagcccgctggactcggccgccggccctgcccgtacgaacaggcgcaggggggagacgggccggaggagcagtgaggggccgcctggtccccggactcagcctccctcctcgccggcctcagtttaccacgtctgaggtcggggggaccccctccgagtcccgcgctgtcttcaaaggcccctgtctcccctccccgacgttggggacgcccctcccagagcccaggtcacctccgggcttccgcagccccctccaaggcggagtggagccttgggaacccctcggccaagcacaggggttcgaaaatacagctgaaaccccgcgggcccttagcacgcgccccagcgccggagcacggtcagggtcttcttgcgacccggcccgctccagatccccacagctttcggccgcggacccgggccgcgtgtgagcgcactttgcacctcctatccccagggtccgccgagagccacgattttttacagaaaatgagcaataaagagattttgtactgtcaaaaa

SEQ ID NO: 3 - protein, 597 aa, homo sapiens
MEAEPPLYPMAGAAGPQGDEDLLGVPDGPEAPLDELVGAYPNYNEEEEERRYYRRKRLGVLKNVLAASAGGMLTYGVYLGLLQMQLILHYDETYREVKYGNMGLPDIDSKMLMGINVTPIAALLYTPVLIRFFGTKWMMFLAVGIYALFVSTNYWERYYTLVPSAVALGMAIVPLWASMGNYITRMAQKYHEYSHYKEQDGQGMKQRPPRGSHAPYLLVFQAIFYSFFHLSFACAQLPMIYFLNHYLYDLNHTLYNVQSCGTNSHGILSGFNKTVLRTLPRSGNLIVVESVLMAVAFLAMLLVLGLCGAAYRPTEEIDLRSVGWGNIFQLPFKHVRDYRLRHLVPFFIYSGFEVLFACTGIALGYGVCSVGLERLAYLLVAYSLGASAASLLGLLGLWLPRPVPLVAGAGVHLLLTFILFFWAPVPRVLQHSWILYVAAALWGVGSALNKTGLSTLLGILYEDKERQDFIFTIYHWWQAVAIFTVYLGSSLHMKAKLAVLLVTLVAAAVSYLRIEQKLRRGVAPRQPRIPRPQHKVRGYRYLEEDNSDESDAEGEHGDGAEEEAPPAGPRPGPEPAGLGRRPCPYEQAQGGDGPEEQ

SEQ ID NO: 4 - DNA, 1777 bp, homo sapiens
gactccggggcgaccgccgcgagtccgcagtagttcgggccatggaggcggagccgccgctctacccgatggcgggggctgcggggccgcagggcgacgaggacctgctcggggtcccggacgggcccgaggccccgctggacgagctggtgggcgcgtaccccaactacaacgaggaggaggaggagcgccgctactaccgccgcaagcgcctgggcgtgctcaagaacgtgctggctgccagcgccgggggcatgctcacctacggcgtctacctgggcctcctgcagatgcagctgatcctgcactacgacgagacctaccgcgaggtgaagtatggcaacatggggctgcccgacatcgacagcaaaatgctgatgggcatcaacgtgactcccatcgccgccctgctctacacacctgtgctcatcaggttttttggaacgaagtggatgatgttcctcgctgtgggcatctacgccctctttgtctccaccaactactgggagcgctactacacgcttgtgccctcggctgtggccctgggcatggccatcgtgcctctttgggcttccatgggcaactacatcaccaggtgagcctggtgggcagcagggcaggaggctggagacctggccaagcctccactttattgccaactttggctgggggaccacaggaagccccttccgccctctgggcctcagtttccccacaccggggctggtctgctcctagctctgggtgcaggacacacaggagtggcacaggtcgggctggggagagccttctctcctttgtggtccaggatggcgcagaagtaccatgagtactcccactacaaggagcaggatgggcaggggatgaagcagcggcctccgcggggctcccacgcgccctatctcctggtcttccaagccatcttctacagcttcttccatctgagcttcgcctgcgcccagctgcccatgatttatttcctgaaccactacctgtatgacctgaaccacacgctgtacaatgtgcagagctgcggcaccaacagccacgggatcctcagcggcttcaacaagacggttctgcggacgctcccgcggagcggaaacctcattgtggtggagagcgtgctcatggcagtggccttcctggccatgctgctggtgctgggtttgtgcggagccgcttaccggcccacggaggagatcgatctgcgcagcgtgggctggggcaacatcttccagctgcccttcaagcacgtgcgtgactaccgcctgcgccacctcgtgcctttctttatctacagcggcttcgaggtgctctttgcctgcactggtatcgccttgggctatggcgtgtgctcggtggggctggagcggctggcttacctcctcgtggcttacagcctgggcgcctcagccgcctcactcctgggcctgctgggcctgtggctgccacgcccggtgcccctggtggccggagcaggggtgcacctgctgctcaccttcatcctctttttctgggcccctgtgcctcgggtcctgcaacacagctggatcctctatgtggcagctgccctttggggtgtgggcagtgccctgaacaagactggactcagcacactcctgggaatcttgtacgaagacaaggagagacaggacttcatcttcaccatctaccactggtggcaggctgtggccatcttcaccgtgtacctgggctcgagcctgcacatgaaggctaagctggcggtgctgctggtgacgctggtggcggccgc

SEQ ID NO: 5 - DNA, 949 bp, somo sapiens
caccggtgtctgaaaaagaaagagcaggaagtatgcatccagatatgtgtgtgtgtactgagctatggtgtgaaatcattcctgactgcgggttatagtcaaaaccccatgaaaagcatcactacagcccacgggtgtgtcagggacacagtgttgtgagccctgggaaggcagggcctgtggccagcactttatcaacactggcgcatgcaccctaagaggcaaagggatttgcattgtccccttacagcgtgggacactgaggtcgccaggggcatggcgactgtaagggacagtgctggatgtgagcctcgcctgcaggaggcggtccaggaaacgtgggtggaggggctggagaaattgagggccgcgtggcccgggaggctcccggaggagggaagggcctatctcagcgaggggcataggcggagaaggtgcggggcgaggcggccgcgggtccgtggcatccctctccttacgcccaggctaagctggcggtgctgctggtgacgctggtggcggccgcggtctcctacctgcggatggagcagaagctgcggcggggcgtggccccgcgccagccccgcatcccgcggccccagcacaaggtgcgcggtgaccgctacttggaggaggacaactcggacgagagcgacgcggagggcgagcatggggacggcgcggaggaggaggcgccgcccccggggcccaggcctggccccgagcccgctggactcggccgccggccctgcccgtacgaacaggcgcaggggggcgacgggccggaggagcagtgaggggccgcctggtccccggactcagcctcgctcctcgccggcctcagtttaccacgtcttaggtcggggggaccccctccgagtcccgcgctgtcttcgaaggcccctgtctcccttcccccacgttggggacgcccctcccagagcccggg